(19) United States
(12) Patent Application Publication
(10) Pub. No.: US 2026/0081030 A1
(43) Pub. Date: Mar. 19, 2026

(54) CLINICAL PATHOLOGIC DATA-FREE AND COMPUTER-AIDED PREDICTION SYSTEM FOR GENETIC VARIATION PATHOGENICITY AND INHERITANCE PATTERN THEREOF AND METHOD THEREOF, AND ION SELECTION SYSTEM AND METHOD THEREOF

(71) Applicants: National Taiwan University Hospital Hsin-Chu Branch, Zhubei City, Hsinchu County (TW); National Tsing Hua University, Hsinchu City (TW)

(72) Inventors: Cheng-Yu Tsai, Zhubei City (TW); Chen-Chi Wu, Zhubei City (TW); Lee-Wei Yang, Hsinchu City (TW)

(73) Assignees: National Taiwan University Hospital Hsin-Chu Branch, Zhubei City, Hsinchu County (TW); National Tsing Hua University, Hsinchu City (TW)

(21) Appl. No.: 19/302,402

(22) Filed: Aug. 18, 2025

(30) Foreign Application Priority Data
Sep. 13, 2024 (TW) 113134991

(51) Int. Cl. G16H 50/30; G16B 5/00; G16B 15/20; G16B 20/50; G16H 50/50

(52) U.S. Cl. CPC G16H 50/30; G16B 5/00; G16B 15/20; G16B 20/50; G16H 50/50

(57) ABSTRACT

A clinical pathologic data-free and computer-aided prediction method for genetic variation pathogenicity and an inheritance pattern thereof includes a molecular dynamics (MD) simulation step, a computing step, a determination step, and a predicted result production step. The MD simulation step is to perform molecular modeling and computational simulations on channel protein configurations, where the channel protein configurations include a wild-type channel or mutated channels. The computing step is to compute the number of ions passing through the wild-type channel and the number of ions passing through the mutated channel, respectively to obtain a first ion number and a second ion number within a predetermined period. The determination step is to determine the class of each mutated channel according to the first ion number and the second ion number. The predicted result production step is to produce a predicted result according to the classes of the mutated channels.

### CROSS-REFERENCE TO RELATED APPLICATION

This non-provisional application claims priority under 35 U.S.C. § 119(a) to Patent Application No. 113134991 filed in Taiwan, R.O.C. on Sep. 13, 2024, the entire contents of which are hereby incorporated by reference.

### BACKGROUND

### Technical Field

A computer-aided prediction system for genetic variation pathogenicity and an inheritance pattern thereof and a method thereof.

### Related Art

Sensorineural hearing loss is a very common disease in clinical practice, and recent studies have confirmed that genetic variation is one of the important causes of idiopathic sensorineural hearing loss. In recent years, it has been found in academia that at least more than two hundred of genes are related to the occurrence of sensorineural hearing loss, that is, hereditary hearing loss.

### SUMMARY

According to the inventor's knowledge, genes that are frequently associated with clinical abnormalities include, for example, GJB2 (Cx26) gene, SLC26A4 (PDS) gene, mitochondrial DNA variation (MT-RNR1 gene) and so on. However, genes and variations related to the disorder of hereditary hearing loss thereof often vary greatly depending on ethnic groups. In addition, some genes may cause different clinical disorders due to different pathogenic variants, and some even may lead to different inheritance patterns. Therefore, the research of hereditary hearing loss becomes complicated and uncertain, or multiple experiments are required to know the inheritance pattern, which results in low efficiency.

In view of this, some embodiments of the present invention provide a clinical pathologic data-free and computer-aided prediction system for genetic variation pathogenicity and an inheritance pattern thereof and a method thereof, and an ion selection system and a method thereof.

According to an embodiment, a clinical pathologic data-free and computer-aided prediction method for genetic variation pathogenicity and an inheritance pattern thereof is provided. The prediction method is suitable for predicting a pathogenic genetic variation related to the disorder of hereditary hearing loss, and the prediction method is executed by a computer arithmetic device. The prediction method includes a molecular dynamics simulation step, a computing step, a determination step, and a predicted result production step.

The molecular dynamics simulation step: performing molecular dynamics simulations on channel protein configurations. The channel protein configurations include a wild-type channel and mutated channels, where the mutated channels include a mutated homomeric channel and a plurality of mutated heteromeric channels.

The computing step: within a predetermined period in the respective computational simulation model, computing the number of ions passing through the wild-type channel to obtain a first ion number, and computing the number of ions passing through the mutated channel to obtain a second ion number.

The determination step: determining a class of each mutated channel according to the first ion number and the second ion number. When the second ion number is less than one-third of the first ion number, the mutated channel is determined as a blocking class. When the second ion number is greater than or equal to half of the first ion number, the mutated channel is determined as a non-blocking class.

The predicted result production step: producing a predicted result according to the classes of the mutated channels.

Additionally, according to an embodiment, a clinical pathologic data-free and computer-aided prediction system for genetic variant of pathogenicity and an inheritance pattern thereof is provided. The prediction system is suitable for predicting a pathogenic genetic variant related to the disorder of hereditary hearing loss. The prediction system includes a simulation module, a processing module and a memory module. The simulation module is configured to execute the molecular dynamics simulation step of the above-mentioned prediction method. The processing module is configured to execute the computing step, the determination step and the predicted result production step of the above-mentioned prediction method. The memory module is configured to store the channel protein configuration and the class of the mutated channel.

In another aspect, according to an embodiment, an ion selection method is provided. The ion selection method is executed by a processing unit, and the ion selection method includes a step S121, a step S122, and a step S123.

Step S121: storing a plurality of position marks for each of a plurality of ions in a time order by selecting each of a plurality of one-dimensional arrays along a first axis of a first two-dimensional tensor. At this step, each of the plurality of position marks for each of the plurality of ions is selected from one element of the group consisting of −a, 0, and b, where a and b are fixed positive integers, −a represents an index of first position, b represents an index of second position, and 0 represents an index of third position.

Step S122: based on a plurality of index values along a second axis of the first two-dimensional tensor, executing the following steps. Step S122-1: for a current index value that is not yet selected, among a plurality of one-dimensional arrays along the second axis of the first two-dimensional tensor, generating an array of net values by subtracting an array indexed with the current index value from an array indexed with the next index value. Step S122-2: setting the array of net values as a one-dimensional array indexed with the current index value along a second axis of second two-dimensional tensor. Step S122-3: repeating the above-mentioned steps S122-1 and S122-2 for arrays of net values until the index values are all selected.

Step S123: in response to the presence of a candidate array of elements with an absolute value of a+b in a plurality of one-dimensional arrays along a first axis of the second two-dimensional tensor, selecting an ion with an index value corresponding to the candidate array from the ions as a candidate ion.

In addition, according to an embodiment, an ion selection system is provided, which includes a processing unit, and the processing unit is configured to execute the ion selection method as described above.

In summary, through the prediction system and the method thereof, a user can predict the pathogenicity of a genetic variant related to hereditary hearing impairment based on physical quantities obtained from computer simulation without preliminary clinical data, and efficiently predict the inheritance pattern of the pathogenicity, and the system is suitable for predicting a pathogenic genetic variant related to the disorder of hereditary hearing loss.

Additionally, according to the ion selection system and the method thereof, resulting specific ions are evaluated using a matrix computation, which may increase the determination efficiency whether the ions permeate through the pore tunnel, and the ion selection system and the method thereof can be applied to the aforementioned prediction system to pick the candidate ions that temporarily passed through a midline of pore tunnel, where these ions have a high probability to entirely pass through the pore tunnel. Therefore, the determination efficiency of the prediction system may be improved.

### BRIEF DESCRIPTION OF THE DRAWINGS

FIG. 1A illustrates a block diagram of a prediction system in some embodiments;

FIG. 1B illustrates a schematic structural diagram of a computer arithmetic device in some embodiments;

FIG. 2 illustrates a flowchart of a prediction method in some embodiments;

FIG. 3 illustrates a top view of a channel protein configuration in a molecular dynamics simulation step in some embodiments, with a dotted chain line meaning the presence a wild-type protein monomer in the channel protein, and a dashed line meaning the presence of a mutated protein monomer in the channel protein, showing that the channel protein configuration can be divided into a wild-type channel, a mutated heteromeric channel and a mutated homomeric channel according to the arrangement and combination of the monomers;

FIG. 4 illustrates a schematic partial cross-sectional view of a channel protein configuration and a phospholipid bilayer in the molecular dynamics simulation in some embodiments, visualizing the channel protein configuration in term of two monomers embedded into the phospholipid bilayer, with dashed lines representing an upper edge, an upper leaflet line, a midline, a lower leaflet line, and a lower edge, respectively;

FIG. 5 illustrates a schematic diagram of the positions and moving trajectory of the ions 20 in the molecular dynamics simulation of channel protein configuration 30 in some embodiments, with dashed lines representing an upper leaflet line, a midline, and a lower leaflet line, respectively;

FIG. 6 illustrates a flowchart of an ion selection method in some embodiments;

FIG. 7 illustrates a schematic diagram of two-dimensional tensor in some embodiments;

FIG. 8 illustrates a schematic diagram of position tensor and transport tensor in some embodiments;

FIG. 9 is a comparison diagram of follow-up hearing outcomes performed in knock-in animal models with different genotypes, showing their progression of hearing outcomes under stimulation of click sounds at sound frequencies of basis, 8 kHz, 16 kHz, and 32 kHz;

FIG. 10A to FIG. 10C are tissue fluorescence staining images of middle turns of Corti's organ from mouse cochleas of knock-in animal models with genotypes Gjb2^{WT/WT}, Gjb2^{WT/V37M} and Gjb2^{V37M/V37M}, respectively; and

FIG. 11 is a comparison diagram of average number of hair cells calculated from the tissue fluorescence staining images of knock-in mouse cochleas with genotype Gjb2^{WT/WT}, Gjb2^{WT/V37M} and Gjb2^{V37M/V37M}.

### DETAILED DESCRIPTION

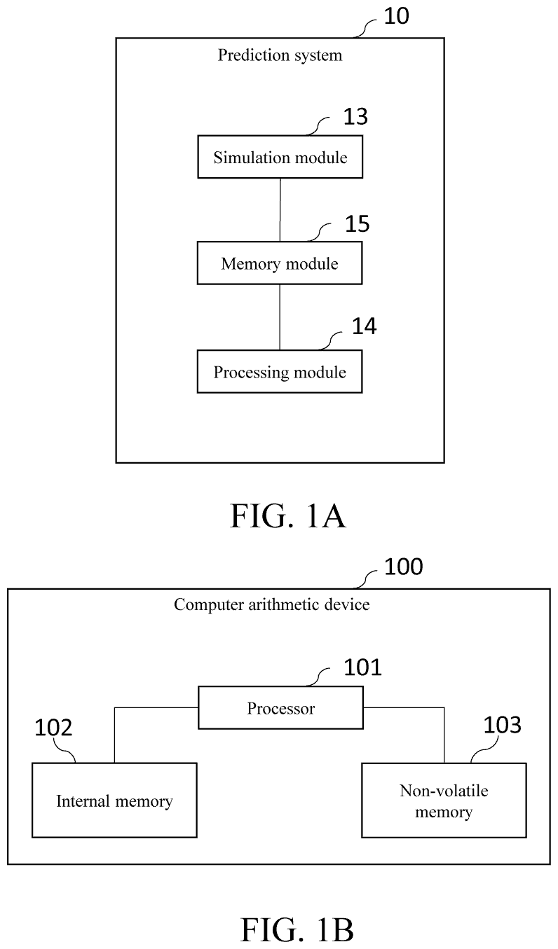

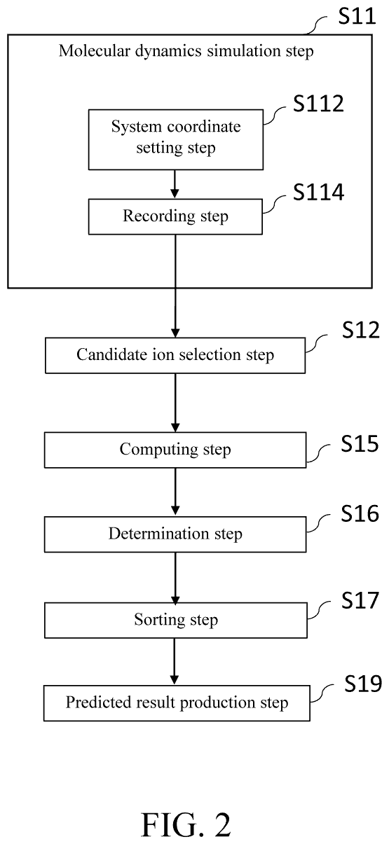

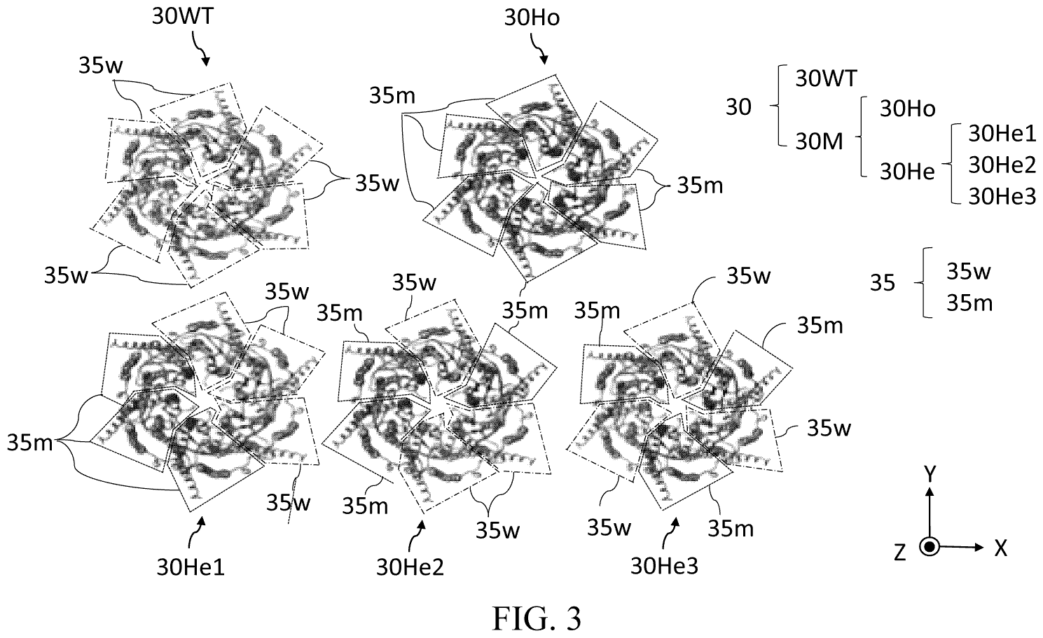

Referring to FIG. 1A, FIG. 1B, FIG. 2 and FIG. 3, FIG. 1A illustrates a block diagram of a prediction system 10 in some embodiments; FIG. 1B illustrates a schematic structural diagram of a computer arithmetic device 100 in some embodiments; FIG. 2 illustrates a flowchart of a prediction method in some embodiments; FIG. 3 illustrates a top view of a channel protein configuration 30 in a molecular dynamics simulation step S11 in some embodiments, with a dotted chain line meaning the presence a wild-type protein monomer 35w in the channel protein, and a dashed line meaning the presence of a mutated protein monomer 35m in the channel protein configuration 30, showing that the channel protein configuration 30 can be divided into a wild-type channel 30WT, a mutated heteromeric channel 30He and a mutated homomeric channel 30Ho according to the arrangement and combination of the monomers 35.

According to an embodiment, a clinical pathologic data-free and computer-aided prediction system (hereinafter referred to as prediction system 10) for genetic variation pathogenicity and an inheritance pattern thereof is provided. The prediction system 10 is executed by a computer arithmetic device 100, and the computer arithmetic device 100 may be, but is not limited to, a computer or a cloud server.

Referring to FIG. 1A, the prediction system 10 includes a simulation module 13, a processing module 14, and a memory module 15. As shown in FIG. 1B, at a hardware level, the computer arithmetic device 100 includes a processor 101, an internal memory 102, and a non-volatile memory 103. The internal memory 102 is, for example, a random-access memory (RAM). The non-volatile memory 103 is, for example, at least one magnetic disc memory and the like. The computer arithmetic device 100 is not limited to the aforementioned hardware, but may also include hardware required for other functions.

The internal memory 102 and the non-volatile memory 103 are configured to store a program, which may include a program code, and the program code includes a computer operating instruction. The internal memory 102 and the non-volatile memory 103 provide instructions and data to the processor 101. The processor 101 reads a corresponding computer program from the non-volatile memory 103 into the internal memory 102 for running, so as to form the prediction system 10 at a logic level (see FIG. 1A). The processor 101 is specifically configured to execute steps recorded in the embodiments below. The processor 101 may be an integrated circuit chip, which has a signal processing capacity. In an implementation process, various methods and steps disclosed in the embodiments below can be completed by an instruction in the form of an integrated logic circuit of hardware or software in the processor 101. The processor 101 may be a general-purpose processor, including a central processing unit (CPU), a digital signal processor (DSP), an application specific integrated circuit (ASIC), a field-programmable gate array (FPGA) or other programmable logic devices, which can implement the methods and steps disclosed in the embodiments below.

The prediction method of various embodiments and how various modules of the prediction system 10 cooperate with each other will be described below in detail with reference to the drawings.

Referring to FIG. 2, the prediction method includes a molecular dynamics simulation step S11, a computing step S15, a determination step S16, and a predicted result production step S19. Also, referring to FIG. 1A together, in the prediction system 10, the simulation module 13 is configured to execute the molecular dynamics simulation step S11. The processing module 14 is configured to execute the computing step S15, the determination step S16, and the predicted result production step S19. The memory module 15 is configured to store the channel protein configurations 30 on which the molecular dynamics simulation step S11 are executed by the simulation module 13, and the classes of the mutated channels 30M determined by the processing module 14.

Referring to FIG. 2 and FIG. 3, the molecular dynamics simulation step S11 is to perform molecular dynamics simulations on the channel protein configurations 30, where the channel protein configurations 30 includes a wild-type channel 30WT and a mutated channels 30M, and the mutated channels 30M includes a mutated homomeric channel 30Ho and a plurality of mutated heteromeric channels 30He. Specifically, each channel protein configuration 30 is a multimer composed of a plurality of monomers 35. When all of each monomer 35 are the wild-type protein monomer 35w, the channel protein configuration 30 is defined as the wild-type channel 30WT. When one of the monomers 35 is not the wild-type protein monomer 35w, or one of the monomers 35 is the mutated protein monomer 35m, the channel protein configuration 30 is defined as the mutated channel 30M. In the composition of the mutated homomeric channel 30Ho, none of the monomers 35 is the wild-type protein monomer 35w. As for the composition of each mutated heteromeric channel 30He, partial monomers 35 are the wild-type protein monomer 35w, and the remaining ones are the mutated protein monomer 35m. The aforementioned “wild-type” represents a protein sequence and structure of an organism in nature, while the aforementioned “mutated” represents a wild-type protein sequence and structure harboring genetic variations. In some embodiments, the wild-type protein monomer 35w is defined as a monomer 35 composed of wild-type protein sequences of species recorded in the academic database Uniprot without any variations, such as human (scientific name: Homo sapiens) wild-type protein sequences (Uniprot ID: P29033) and mouse (scientific name: Mus musculus) wild-type protein sequences (Uniprot ID: Q00977). The mutated protein monomer 35m is defined as a monomer 35 composed of any amino acid substitution—that is, at least one amino acid (i.e., residue) is different from the wild-type protein sequences of the species (i.e., variation).

By way of example, referring to FIG. 3, each individual channel protein configuration 30 is composed of six monomers 35, or is referred to as a hexameric channel configuration. The hexameric channel configurations are categorized into the wild-type channel 30WT and the mutated channels 30M, including homomeric channel 30Ho and heteromeric channels 30He1, 30He2, and 30He3. Each of these channel conformations is constructed from the different combination or arrangement of wild-type protein monomers 35w and mutated protein monomers 35m.

At the molecular dynamics simulation step S11, a plurality of hexameric channel configurations shown in FIG. 3 and their monomers 35 will be further explained. The hexameric channel configuration, composed of six wild-type protein monomers 35w, is defined as the wild-type channel 30WT. The hexameric channel configuration is defined as the mutated channel 30M when incorporating at least one mutated protein monomer 35m. The mutated channels 30M includes the mutated homomeric channel 30Ho and the mutated heteromeric channels 30He. The mutated homomeric channel 30Ho is defined as a mutated channel 30M composed of six mutated protein monomers 35m, all of which have the same variation. The mutated heteromeric channel 30He is defined as a mutated channel 30M composed of some mutated protein monomer 35m and some wild-type protein monomers 35w, where at least one of the six monomers 35 must harbor a variation. The mutated heteromeric channels 30He includes all configurations in various arrangements and combinations of wild-type protein monomer 35w and mutated protein monomer 35m. In some embodiments, referring to FIG. 3, the ratio of the wild-type protein monomer 35w to the mutated protein monomer 35m being 1:1 is taken as an example. The mutated heteromeric channel 30He1 represents the configuration in a composition of three consecutively adjacent wild-type protein monomers 35w and three consecutively adjacent mutated protein monomers 35m. The mutated heteromeric channels 30He, formed of alternating arrangements or combinations of the wild-type protein monomers 35w and mutated protein monomers 35m, are the mutated heteromeric channels 30He2 and 30He3. The patterns of the mutated heteromeric channels 30He of this embodiment is not limited to the one shown in FIG. 3. Depending on the number of the monomers 35 composing the channel protein configuration 30, this embodiment may also include other patterns of composition. For example, the ratio of the wild-type protein monomer 35w to the mutated protein monomer 35m may be, but not limited to, 1:5, 1:2, 2:1, and 5:1. The ratio may be a number ratio or a molar concentration ratio. It should be noted that the number of the monomers 35 in the channel protein configuration 30 described herein is not limited to six. For example, it may be seven (i.e., heptameric channel configuration), eight (i.e., octameric channel configuration), or other integers.

Referring to FIG. 2 and FIG. 3, the computing step S15 is, within a predetermined period in the respective computational simulation model, to compute the number of ions passing through the wild-type channel 30WT to obtain a first ion number, and compute the number of ions passing through each mutated channel 30M to obtain a second ion number. The determination step S16 is to make a decision on the channeling status of each mutated channel 30M according to the first ion number and the second ion number. That is, the determination step S16 is to determine the class of each mutated channel 30M according to the first ion number and the second ion number. At this step, when the second ion number is less than one-third of the first ion number or approaches 0, the mutated channel 30M is determined as the blocking class; and when the second ion number is greater than or equal to half of the first ion number, or is similar to the first ion number, the mutated channel 30M is determined as the non-blocking class. In addition, the predicted result production step S19 is to produce a predicted result according to the classes of the mutated channel 30M. In some embodiments, at the predicted result production step S19, when only the mutated homomeric channel 30Ho, among the mutated channels 30M, is determined as the blocking class, the predicted result indicates autosomal recessive inheritance pattern. By way of example, the first ion number (referred to as “m”) is the number of ions passing through the wild-type channel 30WT in FIG. 3 within the predetermined period set as 10000 nanoseconds, and the second ion number (referred as to “n”) is the number of ions passing through any of the mutated channels 30M in FIG. 3 within 10000 nanoseconds. Given that the first ion number is 6 (i.e. “m”=6), if there is none of ions passing through the mutated homomeric channel 30Ho (i.e. “n”=0), the mutated homomeric channel 30Ho is determined as the blocking class (n equal to 0). If there are three ions passing through any of the mutated heteromeric channels 30He1, 30He2, or 30He3 (i.e. “n”=3), the corresponding individual mutated heteromeric channels can be determined as a non-blocking class (n≥half of m). Since only the mutated homomeric channel 30Ho among the mutated channels 30M is determined as the blocking class, the predicted result produced therefrom is a predicted result of autosomal recessive inheritance.

In addition, in some embodiments at the determination step S16, when second ion number is less than one-third of the first ion number, even equal to 0, the mutated channel 30M is determined as the blocking class; when the second ion number is greater than half of the first ion number, even up to twice, the mutated channel 30M is determined as the non-blocking class.

Thus, without clinical pathologic data, through the prediction system 10 and the method thereof, a user can predict the pathogenicity of a genetic variant based on computer-aided simulated protein configuration results without preliminary clinical proof and effectively predict the inheritance pattern of pathogenicity as a dominant or recessive inheritance pattern. The prediction system enables the prediction on a pathogenic genetic variant related to, but not limited to, the disorder of hereditary hearing loss.

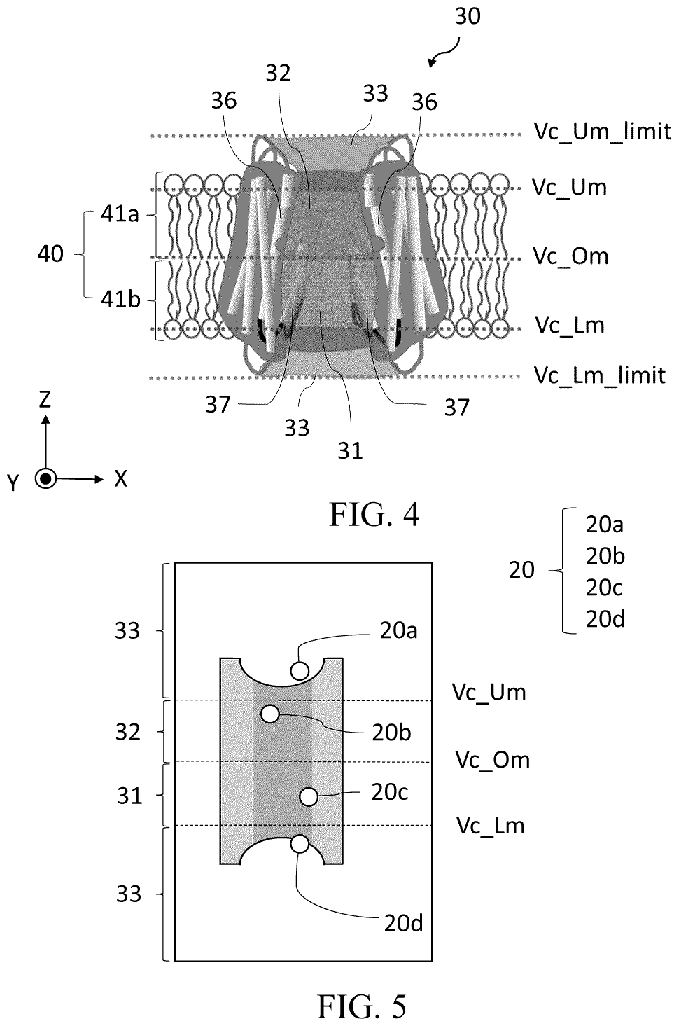

Further, referring to FIG. 4 and FIG. 5, FIG. 4 illustrates a schematic partial cross-sectional view of the channel protein configuration 30 and the phospholipid bilayer 40 in the molecular dynamics simulation in some embodiments, visualizing the channel protein configuration in term of two monomers 35 embedded into the phospholipid bilayer 40, with dashed lines representing an upper edge Vc_Um_limit, an upper leaflet line Vc_Um, a midline Vc_Om, a lower leaflet line Vc_Lm, and a lower edge Vc_Lm_limit, respectively; and FIG. 5 illustrates a schematic diagram of the positions and moving trajectory of the ions 20 in the molecular dynamics simulation of channel protein configuration 30 in some embodiments, with dashed lines representing an upper leaflet line Vc_Um, a midline Vc_Om, and a lower leaflet line Vc_Lm, respectively.

In some embodiments, referring to FIG. 2, the molecular dynamics simulation step S11 further includes a coordinate setting step S112 and a recording step S114. At the coordinate setting step S112, referring to FIG. 4, respective initial average positions of choline in upper leaflet phospholipid 41a and lower leaflet phospholipid 41b of the phospholipid bilayer 40 are defined as an upper leaflet line Vc_Um and a lower leaflet line Vc_Lm, respectively, where the channel protein configuration 30 is located and wrapped in the phospholipid bilayer 40. The phospholipid bilayer 40 may be, but is not limited to, 1,2-dioleoyl-sn-glycero-3-phosphocholine (DOPC), which has two layers of phospholipids 41a and 41b. Taking the schematic cross-sectional view shown in FIG. 4 as an example, in the phospholipid bilayer 40, the average position of choline in upper leaflet phospholipids 41a is defined as the upper leaflet line Vc_Um, and the average position of choline in the lower leaflet phospholipids 41b is defined as the lower leaflet line Vc_Lm.

In some embodiments, at the molecular dynamics simulation step S11, in molecular dynamics simulations, the upper leaflet line Vc_Um is the average position of nitrogen atom coordinates of each choline in the phospholipids 41a, and the lower leaflet line Vc_Lm is the average position of nitrogen atom coordinates of each choline in the phospholipids 41b. Under a sampling time interval (100 picoseconds), the whole system makes a calibration of normalization for system coordinates based on the positions of Vc_Um and Vc_Lm measured from initial system coordinates for the standard in subsequent measurement and analysis of physical quantities in an embodiment.

The recording step S114 is to record position changes of a plurality of ions 20 in each channel protein configuration 30 to obtain a plurality of moving trajectories within a predetermined simulation period of the molecular dynamics simulation. Take the channel protein configuration 30 of FIG. 5 as an example. Within the rectangular periodic boundary simulation system shown in FIG. 5, a position range between the lower leaflet line Vc_Lm and the midline Vc_Om is a first tunnel partition (position 31), and a position range between the upper leaflet line Vc_Um and the midline Vc_Om is a second tunnel partition (position 32). The remaining area of the rectangular periodic boundary simulation system excluding from the positions 31 and 32 is a position 33, which represents the non-membrane region. Take the channel protein configuration 30 and ions 20 shown in FIG. 5 as an example. In the molecular dynamics simulation system, within the predetermined simulation period of 10000 nanoseconds (the length of the simulation timescale is not limited in this embodiment), the positions of individual ion 20 at different tunnel partitions within the channel protein configuration 30, marked as 20a, 20b, 20c, and 20d that are recorded at different snapshots (i.e., system static coordinates at certain time point with time interval of 1 nanosecond) of individual system to obtain an overall moving trajectory of each ion 20 in the whole predetermined simulation period.

Next, referring to FIG. 3 and FIG. 5 together, in some embodiments, in the computing step S15, the first ion number and the second ion number respectively represent numbers of ions 20 whose trajectories intersect both the upper leaflet line Vc_Um and the lower leaflet line Vc_Lm of the corresponding wild-type channel 30WT and the mutant channel 30M. The ions 20 completely traverse the respective channel protein configuration. By way of example, taking the non-membrane region as an initial position (i.e. the position 33), when an ion moving trajectory completely intersects with both the upper leaflet line Vc_Um and the lower leaflet line Vc_Lm from the initial position, the corresponding ions 20 are determined as permitted ions. Either first ion number or the second ion number are computed in this same way. In FIG. 5, the illustrative ion 20 departs from a position 33 (ion 20a), through a position 32 (ion 20b) and a position 31 (ion 20c), and finally reaches a position 33 (ion 20d) in a sequential movement within a continuous time period. This ion 20 will be recorded with its membrane-penetrating moving trajectory, encompassing the order of position 33 close to the upper leaflet line Vc_Um, the position 32, the position 31, and the position 33 close to the lower leaflet line Vc_Lm. Alternatively, a reverse trajectory (i.e. the route in the position order of ion 20d, 20c, 20b, and 20a) intersects with the upper leaflet line Vc_Um, the midline Vc_Om, and the lower leaflet line Vc_Lm, and thus, the ions 20 are determined as permitted ion. On the contrary, it is assumed that the recorded moving trajectory of the ion 20 within a period only intersects with the upper leaflet line Vc_Um or/and the midline Vc_Om (without intersecting with the lower leaflet line Vc_Lm), or only intersects with the lower leaflet line Vc_Lm or/and the midline Vc_Om (without intersecting with the upper leaflet line Vc_Sm), then the ions 20 are not classified as the permitted ions. Each ion 20 is allowed to have a plurality of membrane-penetrating moving trajectories within the whole predetermined simulation period. Once individual ions 20 finally reaches to the opposite position 33 (e.g., positive membrane penetration into the non-membrane region at the position of ion 20a or 20d) or returns to the initial counterpart (e.g., negative membrane penetration) within a continuous time period, this round of recording for its moving trajectory will be ended. In the subsequent simulation time, when any individual ions 20 initially or once again enter the position 31 (e.g., the position of ion 20c) or the position 32 (e.g., the position of ion 20b) departed from position 33, a new round of recording for its membrane-penetrating moving trajectory will be initialized again.

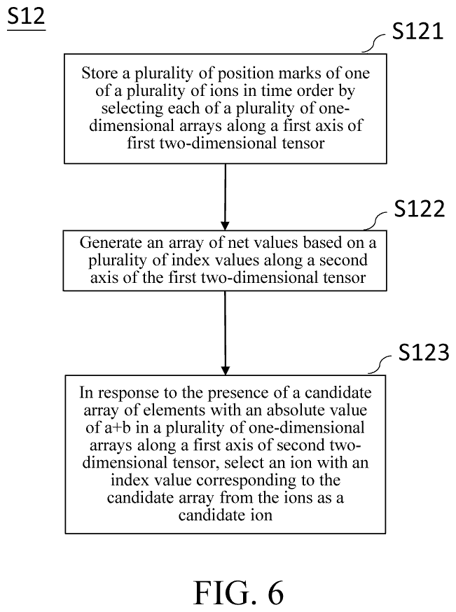

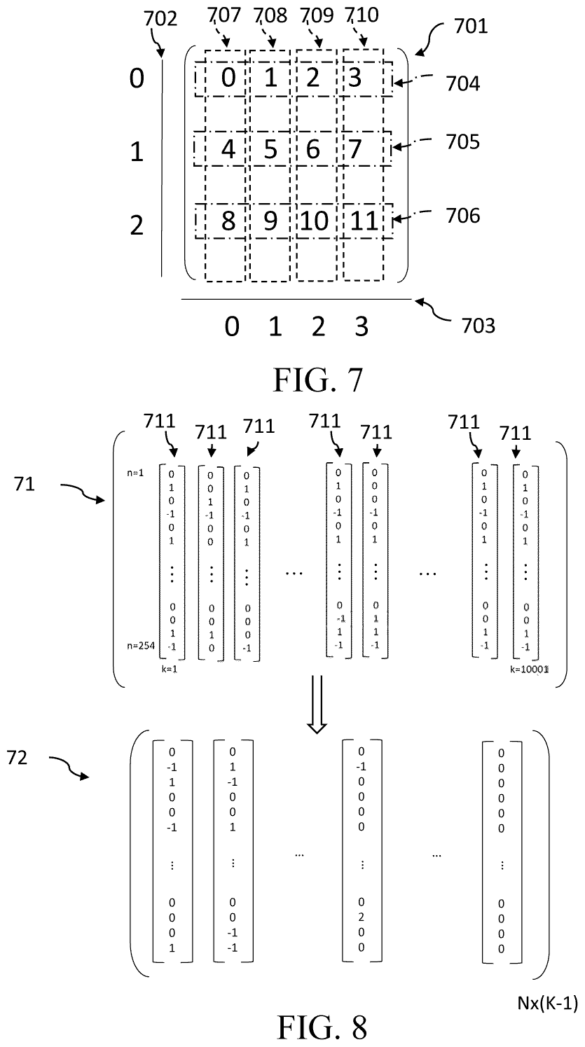

In another aspect, referring to FIG. 2, FIG. 6, FIG. 7 and FIG. 8, FIG. 6 illustrates a flowchart of an ion selection method in some embodiments; FIG. 7 illustrates a schematic diagram of two-dimensional tensor 701 in some embodiments; and FIG. 8 illustrates a schematic diagram of position tensor 71 and transport tensor 72 in some embodiments. In some embodiments, an ion selection system is provided, which includes a processing unit for executing an ion selection method. The processing unit may be the processor 101 of FIG. 1B. In addition, the ion extraction method is suitable for the prediction system described in the embodiments herein. Referring to FIG. 6, the ion selection method includes a candidate ion selection step S12, which incorporates step S121, step S122, and step S123.

Referring to the two-dimensional tensor 701 shown in FIG. 7 first for illustration, the two-dimensional tensor 701 is constructed based on a 0^{th} axis (e.g., an axis 702 shown in FIG. 7) and a 1^{st} axis (e.g., an axis 703 shown in FIG. 7). There is at least one index value along each axis (e.g., in FIG. 7, a set of index values 0, 1, and 2 along the 0^{th} axis, and another set of index values 0, 1, 2, and 3 along the 1^{st} axis). The integer values are used for indexing elements in the two-dimensional tensor 701. For example, in FIG. 7, an element 1 of the two-dimensional tensor 701 can be obtained from the index value 0 along the 0^{th} axis and the index value 1 along the 1^{st} axis (marked as T[0][1]=1, where T is a representative symbol of the two-dimensional tensor 701). Each axis of the two-dimensional tensor 701 is based on its respective index value and has at least one one-dimensional tensor (or referred to as one-dimensional array) corresponding to the index value of each axis. For example, in FIG. 7, the two-dimensional tensor 701 has individual one-dimensional arrays 704, 705, and 706 along the 0^{th} axis, where the one-dimensional array 704 is indexed with value 0, the one-dimensional array 705 is indexed with value 1, and the one-dimensional array 706 is indexed with value 2. In the same FIG. 7, the two-dimensional tensor 701 along the 1^{st} axis has individual one-dimensional arrays 707, 708, 709, and 710, where the one-dimensional array 707 is indexed with value 0, the one-dimensional array 708 is indexed with value 1, the one-dimensional array 709 is indexed with value 2, and the one-dimensional array 710 is indexed with value 3. It is to be noted that the structure of the two-dimensional tensor 701 is the same as that of a matrix. The index values of one-dimensional array on the 0^{th} axis of the two-dimensional tensor 701 forms the rows of the matrix, and the index values of one-dimensional array on the 1^{st} axis of the two-dimensional tensor 701 forms the columns of the matrix.

Referring to FIG. 5 and FIG. 8, in some embodiments, in step S121, the processing unit stores a plurality of position marks for each of the ions 20 in a time order by selecting each of a plurality of one-dimensional arrays on a first axis of a first two-dimensional tensor. Each position mark of each ion 20 is selected from one element of a mark set {“−a”, “0”, “b” }. In some embodiments, “a” and “b” are fixed positive integers, “−a” represents an index of the position 31, “b” represents an index of the position 32, and “0” represents an index of the position 33. It is to be noted that before executing step S121, the processing unit may load the moving trajectories of a plurality of ions 20 recorded in the recording step S114.

By way of example, referring to FIG. 2, FIG. 5 and FIG. 8, at the molecular dynamics simulation step S11, the moving trajectories of a plurality of (e.g., 254) ions 20 are simulated and recorded within the predetermined period (e.g., 10000 nanoseconds), and the position 31, the position 32, and the position 33 shown in FIG. 5 are defined at the system coordinate setting step S112 in the above-mentioned embodiments. In this embodiment, when the ions 20 are located in the position 31, which is the position of ion 20c shown in FIG. 5, the position of the ions 20 is marked as “−1” (i.e., in step S121, “a” is selected as 1). When the ions 20 are located in the position 32, which is the position of ion 20b shown in FIG. 5, the position of the ions 20 is marked as “1” (i.e., in step S121, “b” is selected as 1). When the ions 20 are located in the positions 33, which is the positions of ions 20a and 20d shown in FIG. 5, the position of the ions 20 is marked as “0” (i.e., the value 0 in step S121). In FIG. 8, a plurality of positions for one of the ions 20 are recorded in a simulation time order with a plurality of one-dimensional arrays on a 0^{th} axis of the position tensor 71. At this time, each one-dimensional array 711 on a 1^{st} axis of the position tensor 71 represents the position of each ion 20 at a specific time point.

Taking the embodiment illustrated in FIG. 8 as an example, a plurality of one-dimensional arrays on the 0^{th} axis (serving as the first axis of the first two-dimensional tensor) of the position tensor 71 (serving as the first two-dimensional tensor) store a plurality of positions for one of a plurality of (e.g., 254) simulated ions in a simulation time order. For example, the one-dimensional array indexed with value 0 along the 0^{th} axis of the position tensor 71 stores the position of the first ion (marked as n=1, where n is an ion index) at each time point, and the one-dimensional array indexed with value 1 along the 0^{th} axis of the position tensor 71 stores the position of the second ion (marked as n=2) at each time point, and so on, until the positions of all 254 simulated ions at each time point are recorded. It is to be noted that n=254 marked in FIG. 8 indicates that a one-dimensional array with index value up to 253 along the 0^{th} axis of the position tensor 71 stores the position of a total of 254 ions at each time point). At this time, each one-dimensional array (i.e., a plurality of one-dimensional arrays 711 in FIG. 8) on the 1^{st} axis (serving as a second axis of the first two-dimensional tensor) of the position tensor 71 represents the position of all simulated ions at a specific time point. For example, the one-dimensional array indexed with value 0 along the 1^{st} axis of the position tensor 71 is the one-dimensional array 711 that stores the position of all simulated ions at the time point of 0^{th} ns (i.e., initial coordinates before simulation; marked as k=1, where k is the time index of interval in the unit of nanosecond). The one-dimensional array indexed with value 1 along the 1^{st} axis of the position tensor 71 is the one-dimensional array 711 that stores the position of all simulated ions at the time point of 1^{st} ns (marked as k=2, where k is the time index of interval in the unit of nanosecond), and so on. It is to be noted that k=10001 marked in FIG. 8 indicates that the one-dimensional array corresponding to the index value 10000 along the 1^{st} axis of the position tensor 71 stores the position of all ions at the time point of 10000^{th} ns. Thus, the position tensor 71 records the position marks for each of 254 ions in time order over 10000 ns. The 0^{th} axis of the position tensor 71 (serving as the first axis of the first two-dimensional tensor) has N dimensions, and the 1^{st} axis of the position tensor 71 (serving as the second axis of the first two-dimensional tensor) has K dimension, where N=254, and K=10001.

In step S122, based on a plurality of index values along the second axis of the first two-dimensional tensor, an array of net values is generated. In some embodiments, the step S122 includes based on a plurality of index values of a second axis of the first two-dimensional tensor, executing the following steps:

For a current index value that is not yet selected, among a plurality of one-dimensional arrays along the second axis of the first two-dimensional tensor, an array of net values is generated by subtracting an array indexed with the current value from another array indexed with the next value. The array of net values is set as a one-dimensional array indexed with the current value along a second axis of a second two-dimensional tensor. The above-mentioned steps for arrays of net values are repeated until all the index values are selected. It is to be noted that if the current index value is the last index value of the second axis of the first two-dimensional tensor, there will be no next index value. At this time, the processing unit continues to select a next current index value, and if there is no next current index value, it indicates that all the index values along the second axis of the first two-dimensional tensor are selected.

It will be illustrated with the position tensor 71 illustrated in FIG. 8 taken as an example. If the current index value is 0, then the processing unit subtracts an array (i.e., (0 1 0 −1 0 1 . . . 0 0 1 −1)^{T}) indexed with value 0 from an array (i.e., (0 0 1 −1 0 0 . . . 0 0 1 0)^{T}) indexed with value 1 along the 1^{st} axis of the position tensor 71 (serving as the second axis of the first two-dimensional tensor) to produce an array of net values ((0 −1 1 0 0 −1 . . . 0 0 0 1)^{T}). The processing unit sets the array of net values as a one-dimensional array indexed with value 0 along a 1^{st} axis (serving as the second axis of the second two-dimensional tensor) of the transport tensor 72 (serving as the second two-dimensional tensor). The above-mentioned step is to subtract the one-dimensional array 711 at (k−1)^{th} ns from the one-dimensional array 711 at kth ns to produce an array of net values, where a set of the arrays of net values forms the transport tensor 72. At this time, a 0^{th} axis (serving as a first axis of the second two-dimensional tensor) of the transport tensor 72 has N dimensions, and the 1^{st} axis (serving as the second axis of the second two-dimensional tensor) of the transport tensor 72 has K−1 dimensions, where N=254, and K=10001.

In step S123, in response to the presence of a candidate array of elements with an absolute value of a+b in the plurality of one-dimensional arrays on the first axis of the second two-dimensional tensor, an ion 20 with an index value corresponding to the candidate array is selected from the ions 20 as one of the candidate ions. In the embodiment illustrated in FIG. 8, the candidate array is an array of elements with an absolute value of 2 (i.e., the element value is 2 or is −2).

Thus, according to the ion selection system and a method thereof, the resultant specific ions are evaluated using a matrix computation, which can increase the determination efficiency for the ions 20 whether or not permeate through the tunnel of the channel protein.

It is to be noted that when selecting a plurality of index values along the second axis of the first two-dimensional tensor, the index values may be selected in ascending order, that is, the processing unit takes the index value 0 in the current step, followed by the index value 1 in the next step, and so on.

It is to be noted that while in the embodiment of FIG. 8, the 0^{th} axis of the position tensor 71 is taken as the first axis of the first two-dimensional tensor. Alternatively, the 1^{st} axis of the position tensor 71 may be taken as the first axis of the first two-dimensional tensor, which will not be limited in the present invention. In addition, the aforementioned “a” and “b” may be selected as other values, which will not be limited in the present invention.

In some embodiments, the processing module 14 of FIG. 1A executes the candidate ion selection step S12, where the candidate ion selection step S12 is the ion selection method of the above-mentioned embodiment. Next, in the computing step S15, it is determined whether a corresponding candidate ion is a permeated ion according to the moving trajectory of the candidate ion selected in the candidate ion selection step S12. Thus, through the candidate ion selection step S12, the ions 20 in the simulation system are first screened to find out the candidate ions recorded to pass through the midline Vc_Om. The candidate ions have a higher probability of passing through the tunnel of the whole channel protein configuration 30, and then the moving trajectories of the candidate ions are analyzed, thereby improving the efficiency of the prediction system 10.

Referring to FIG. 2, in some embodiments, after the molecular dynamics simulation step S11, the prediction method further includes a sorting step S17. At the sorting step S17, the mutated heteromeric channels 30He are sorted based on conformational potential energy obtained from the molecular dynamics simulation in which each mutated heteromeric channel 30He reaches conformation equilibrium. One of the mutated heteromeric channels 30He with the lowest conformational potential energy is defined as a major mutated heteromeric channel. Moreover, at the predicted result production step S19, when both of the mutated homomeric channel 30Ho and the major mutated heteromeric channel are determined as the blocking class, the predicted result indicates autosomal dominant inheritance. The aforementioned conformational potential energy may be, but is not limited to, average conformational potential energy or final conformational potential energy.

Referring to FIG. 3, in some embodiments, take the average conformational potential energy as an example. In molecular dynamics simulation within 10000 nanoseconds, after the protein conformation reaches at least dynamic equilibrium, the conformational potential energy at each snapshot (with a snapshot time interval of 1 nanosecond) at the final period of the simulation (e.g., 1000 nanoseconds) is taken to work out the average configurational potential energy. According to Boltzmann relation:

$$P = exp ( - {E}_{conf} / {k}_{B} T )$$

E_{conf} is the average conformational potential energy, k_{B} is the Boltzmann constant, T is the Boltzmann temperature, and P is the conformation existence probability. The simulation result shows that the average conformational potential energy E_{conf} of the mutated heteromeric channel 30He2 at the final period is the highest, followed by that of the mutated heteromeric channel 30He3, and finally that of the mutated heteromeric channel 30He1. Therefore, since the average conformational potential energy E_{conf} obtained from each of the mutated heteromeric channels 30He1 and 30He3 is lower than the average conformational potential energy E_{conf} of the mutated heteromeric channel 30He2, the conformation existence probability of both mutated heteromeric channels 30He1 and 30He3 are higher than that of 30He2. Therefore, both of the mutated heteromeric channels 30He1 and 30He3 are defined as the major mutated heteromeric channel. Next, if both of the mutated heteromeric channels 30He1 and 30He3 are determined as the blocking class, same as the mutated homomeric channel 30Ho determined as the blocking class, the produced predicted result indicates the autosomal dominant inheritance.

Referring to FIG. 3 and FIG. 4, in some embodiments, the channel protein configurations 30 are categorized as at least one of connexin families. The genes of the channel protein configuration 30 may include, but are not limited to, GJB2 gene (connexin26, Cx26), GJB3 gene (connexin31, Cx31), and GJB6 (connexin30, Cx30) gene. Channel proteins include Cx26 connexin, Cx30 connexin, Cx31 connexin, Cx26/Cx30 heteromeric or heterotypic connexin composed of Cx26 and Cx30 monomers, and Cx26/Cx31 heteromeric or heterotypic connexin composed of Cx26 and Cx31 monomers.

Referring to FIG. 4, in some embodiments, in system coordinate setting step S112, segments of each channel protein configuration 30 located within the upper leaflet line Vc_Um and the lower leaflet line Vc_Lm are defined as channel portions, segments located within the upper leaflet line Vc_Um and the upper edge Vc_Um_limit are defined as extracellular portion, and segments located within the lower leaflet line Vc_Lm and the lower edge Vc_Lm_limit are defined as intracellular portion. The gene of the channel protein configuration 30 is GJB2, whose encoded protein is the monomer 35 known as Cx26 connexin. The Cx26 connexin includes an N-terminal helice (referred to as “NTH”) domain 37 and a plurality of transmembrane (referred to as “TM”) domains, where the TM domain closest to the NTH domain 37 is the first TM domain 36. Both NTH domain 37 and the first TM domain 36 constitute the motif, which is the major component of inner edge in the tunnel regions 31 and 32 of the hexameric channel protein, as shown in FIG. 4. Additionally, the schematic diagram of the hexameric channel configuration composed of the wild-type Cx26 monomers is for the wild-type channel 30WT as shown in FIG. 3, and the schematic diagram of the hexameric channel configuration composed of the mutated Cx26 monomers is for any of the mutated channels 30M as shown in FIG. 3. In some embodiments, the monomer 35 composing the mutated channel 30M harbors at least one mutated residue. By way of specific examples, in some embodiments, the monomer 35 of the mutated channel 30M is mutated into isoleucine or methionine from valine at the 37^{th} residue of the first TM domain 36 in FIG. 4. It is to be noted that the position of the mutated residue may be, but is not limited to, the NTH domain 37 and the first TM domain 36 of the channel protein configuration 30, or may be second, third, and fourth TM domain or a C-terminal loop.

In another aspect, in the embodiments below, C57BL/6 transgenic mice were used for constructing knock-in mice animal models with Gjb2 gene p.V37M variation to demonstrate whether the predicted result of the inheritance pattern of the pathogenicity of the aforementioned embodiment is consistent with the inheritance pattern results obtained from animal experiments.

### Construct Membrane-Embedded Protein Cx26 Model by Computer Simulation

Since a protein tertiary crystal structure (PDB ID: 2ZW3) of human connexin 26 (hereinafter referred to as hCx26) in the RCSB Protein Data Bank (hereinafter referred to as PDB) with missing protein segments, encompassing the loop of 110-124 residues and N-terminal amino acid residues (Met1), the lost fragments of the original crystal structure were modeled and repaired using the academic platform SWISS-MODEL (https://swissmodel.expasy.org/) and open source software PyMOL (version 2.3.3, Schrödinger, LLC) according to the wild-type protein sequence (UniProt ID: P29033) of hCx26. Through the above approach, a hCx26 homology monomer configuration without any missing fragments was reconstructed, followed by the repeated superimposition of homology monomer to each monomer of crystal structure 2ZW3 to finally constitute a wild-type human Cx26 hexameric hemichannel (hereinafter referred to as WT-hCx26), whose configuration was similar to that of the wild-type channel 30WT shown in FIG. 3. According to the academic software PROPKA (version 3.1) platform, the protein protonation state of WT-hCx26 in a neutral pH environment was calibrated. Next, a rectangular periodic boundary simulation framework of 14×14×19 nm³ was generated using the academic platform CHARMM-GUI (https://www.charmm-gui.org/), and WT-hCx26 was embedded into a phospholipid bilayer, where this membrane was composed of 444 1,2-dioleoyl-sn-glycero-3-phosphocholine (hereinafter referred to as DOPC) molecules. The ionic condition was set as a 150 mM potassium chloride in the aqueous solution for simulating the high potassium concentration environment of lymphatic fluid in the cochlea. Parameters (such as tilt angle and penetration depth) related to the position and direction of membrane proteins embedded into the phospholipid bilayer were constructed using “Orientations of Proteins in Membranes (OPM)” in a built-in database of the CHARMM-GUI platform by employing the Positioning of Proteins in Membrane (PPM, version 2.0). Additionally, based on the aforementioned method and a WT-hCx26 model as template, a mutant with homomeric V37I variant of human Cx26 hexameric hemichannel (hereinafter referred to as V37I-hCx26) model was constructed, and its configuration was similar to the mutated homomeric channel 30Ho shown in FIG. 3.

Similarly, a wild-type mouse Cx26 hemichannel (hereafter termed as WT-mCx26, with a structure similar to that of a wild-type channel 30WT shown in FIG. 3) and a mutant with V37M variant of mouse Cx26 hexameric hemichannel (hereafter termed as V37M-mCx26, with a structure similar to that of a mutated channel 30M shown in FIG. 3) were built using the SWISS-MODEL platform based on the protein sequence (UniProt ID: Q00977) of mouse connexin 26 (hereinafter referred to as mCx26) and the crystal structure (PDB ID: 2ZW3) of hCx26 in the same way as the aforementioned model construction process. The phospholipid bilayer was constructed using the same 444 DOPC molecules, and a reconstructed mCx26 protein was embedded into the membrane to construct a rectangular periodic boundary simulation system with the same high potassium concentration environment (150 mM potassium chloride).

Four models were constructed for the aforementioned V37M-mCx26. The first model was composed of six V37M mutated monomers, where each mutated monomer was the mutant with the same V37M variant, forming the mouse Cx26 homomeric channel (termed as V37M-mCx26 homomer, with a structure similar to that of the mutated homomeric channel 30Ho shown in FIG. 3). The latter three models were composed of three wild-type monomers and three V37M mutated monomers, respectively, in different arrangements and combinations, where the three mutated channel protein formed a type I mutant with V37M variant in mouse Cx26 heteromeric channel (hereafter termed as V37M-mCx26 heteromer-1, with a structure similar to that of the mutated heteromeric channel 30He1 shown in FIG. 3), a type II mutant with V37M variant in mouse Cx26 heteromeric channel (hereafter termed as V37M-mCx26 heteromer-2, with a structure similar to that of the mutated heteromeric channel 30He2 shown in FIG. 3), and a type III mutant with V37M variant in mouse Cx26 heteromeric channel (hereafter termed as V37M-mCx26 heteromer-3, with a structure similar to that of the mutated heteromeric channel 30He3 shown in FIG. 3). It is to be noted that all of mCx26, WT-mCx26, V37M-mCx26 homomer, V37M-mCx26 heteromer-1, V37M-mCx26 heteromer-2 and V37M-mCx26 heteromer-3 are the channel protein configuration 30. Moreover, all of V37M-mCx26 heteromer-1, V37M-mCx26 heteromer-2, and V37M-mCx26 heteromer-3 were composed of wild-type protein monomers 35w and V37M mutated protein monomers 35m in a ratio of 1:1, forming the protein configuration of hexameric hemichannel in different arrangements and combinations for in silico simulations.

### Molecular Dynamics Simulation

Using the GROMACS software suite and the Martini force field, approximately 4 μs of molecular dynamics simulation was performed in individual simulation model. The simulation condition was set as follows: the pressure was 1 bar; the temperature was Kelvin temperature of 310 K; the cut-off value of both Coulomb force and Van der Waals force was 1.1 nm; an NPT ensemble was set with the fixed number of particles, pressure, and temperature that were regulated using Parrinello-Rahman barostat and Berendsen thermostat; a system equilibrium phase of at least 30 ns for the production phase of 4 ρs simulation time. Additionally, all simulated trajectories were computed at a time interval of 20 fs per step, and all coordinate values were recorded once at a rate of 1 ns/frame. In addition, the mean of the average vertical coordinates of the upper and lower choline groups of a phospholipid bilayer per frame was taken as the vertical coordinate center of the whole rectangular periodic boundary simulation, which was used for the systematic vertical coordinate calibration in each frame.

In molecular dynamics simulation, the prediction system 10 for the inheritance pattern of the pathogenicity was executed based on the steps shown in FIG. 2, and various physical quantities are measured as presented in the following tables.

TABLE 1
Number of Permeated Potassium Ions

| Models | Number of Permeated Potassium Ions |
|---|---|
| WT-hCx26 | 3 |
| V37I-hCx26 | 0 |
| WT-mCx26 | 6 |
| V37M-mCx26 homomer | 0 |
| V37M-mCx26 heteromer-1 | 3 |
| V37M-mCx26 heteromer-2 | 0 |
| V37M-mCx26 heteromer-3 | 4 |

Table 1 shows the prediction system 10 performs the steps shown in FIG. 2. After the candidate ions were obtained in step S12, the number of permeated potassium ions in each model under 4 μs simulation was computed (that is, step S15). Next, according to step S16, its determination result is that V37M-mCx26 homomer and V37M-mCx26 heteromer-2 are determined as blocking channel, and V37M-mCx26 heteromer-1 and V37M-mCx26 heteromer-3 are a non-blocking channel. Accordingly, the predicted result produced in step S19 is the predicted result of autosomal recessive inheritance.

TABLE 2
Final conformational potential energy

| | Final Conformational Potential |
|---|---|
| Models | Energy E_{conf} (kJ/mol) |
| WT-mCx26 | −7.17 × 10⁴ |
| V37M-mCx26 homomer | −6.49 × 10⁴ |
| V37M-mCx26 heteromer-1 | −6.54 × 10⁴ |
| V37M-mCx26 heteromer-2 | −6.41 × 10⁴ |
| V37M-mCx26 heteromer-3 | −6.50 × 10⁴ |

Table 2 shows the final conformational potential energy E_{conf} in each model under 4 ρs simulation. The lower potential energy E_{conf} mean conformation existence probability. Given the order of potentials (WT-mCx26<V37M-mCx26 heteromer-1<V37M-mCx26 heteromer-3<V37M-mCx26 homomer<V37M-mCx26 heteromer-2), the top three conformers (WT-mCx26, V37M-mCx26 heteromer-1, V37M-mCx26 heteromer-3) determined as non-blocking class shows lower potentials than the other two models (V37M-mCx26 homomer, V37M-mCx26 heteromer-2) determined as blocking class, predicted to predominate the channel protein conformation proportion. This result may show that in an unaffected carrier (i.e., an individual with pathogenic variant of heterozygote but without hearing loss). Since two types of Cx26 monomers (i.e., wild-type monomer 35w and mutated monomer 35m) can arbitrarily compose a wild-type or any hexameric channel conformation in vivo, when the hexameric channel conformation in non-blocking class occupy predominant proportion due to their lower energy than any mutated hexameric channel conformation in blocking class, the carrier's channel function is expected to normally maintained, without affecting normal hearing. In contrast, in an affected patient, i.e., an individual with pathogenic variant of homozygote and hearing loss, there are only mutated homomeric channel proteins with a poor channel function determined as blocking class in vivo, which leads to hearing impairment. Additionally, V37M-mCx26 heteromer-1 has the lowest final conformational potential energy among mutated heterodimeric hexamers. Therefore, according to the sorting step S17, its determination result is that V37M-mCx26 heteromer-1 serves as the main mutated heteromeric channel.

TABLE 3
Average Number of Water Molecules
in Channel in Simulation Time

| Models | Average Number of Water Molecules |
|---|---|
| WT-hCx26 | 51.83 ± 0.19 |
| V37I-hCx26 | 27.61 ± 0.12*** |
| WT-mCx26 | 67.22 ± 0.60 |
| V37M-mCx26 homomer | 47.67 ± 0.54*** |
| V37M-mCx26 heteromer-1 | 62.09 ± 0.68*** |
| V37M-mCx26 heteromer-2 | 47.84 ± 0.35*** |
| V37M-mCx26 heteromer-3 | 70.50 ± 0.60*** |

Table 3 shows the average number of water molecules in each model taken from multiple snapshots in the interval of 10 ns/frame under 4 s simulation, computing the mean of water molecules located in the channel portion of each snapshot. The average number of water molecules in V37M-mCx26 heteromer-2 is less than that of WT-mCx26, while the average number of water molecules in V37M-mCx26 heteromer-1 and V37M-mCx26 heteromer-3, respectively, are similar to the average number of the water molecules in WT-mCx26. The results in Table 3 indicated that low average numbers of water molecules (i.e., narrow tunnel volume) in both V37M-mCx26 homomer and V37M-mCx26 heteromer-2, which are determined as blocking channels, are related to their reduced pore permeability. While, V37M-mCx26 heteromer-1 and V37M-mCx26 heteromer-3 suggesting high average numbers of water molecules (i.e., broad tunnel volume), are related to their normal pore permeability determined as non-blocking channels. Combined with Table 1 and Table 3 results, these results demonstrate that the trend of the number of permeated potassium ions in each model is highly correlated to that of the average number of water molecules in the channel, that is, the determination result obtained in the determination step S16 is based on both trends of permeated potassium ions and average number of water molecules.

Additionally, it is to be noted that the Table 3 lists the average number of water molecules in each model with standard error of the mean (SEM). The p-value for significant differences was computed using Student's t-test, which was the result of the mutated channels 30M of each model compared with the corresponding wild-type channels 30WT (i.e., WT-hCx26 or WT-mCx26) in each data set (human model or mouse model), where * represents p-value less than or equal to 0.05, ** represents p-value less than or equal to 0.01, and * represents p-value less than or equal to 0.001.

In addition, in the molecular dynamics simulation of each hexameric hemichannel, based on the Martini force field, the affinity, i.e., non-bonding energy, between the NTH domain 37 and the first TM domain 36 (as shown in FIG. 4) of each monomer was computed according to electrostatic and Van der Waals forces. Regarding the affinity measured in each model, in the WT-mCx26 model the affinity between Val (V) at the 37^{th} residue and Trp (W) at the 3^{rd} residue [V37, W3]^{WT} was −83.43±0.16 kJ mol⁻¹, and the affinity between Met (M) at the 34^{th} residue and Trp at the 3^{rd} residue [M34, W3]^{WT} was −103.46±0.17 kJ mol⁻¹. The affinity in the V37M-mCx26 homomer model between mutated Met at the 37^{th} residue and Trp at the 3^{rd} residue [M37, W3]V37M was −98.25±0.2 kJ mol⁻¹, and the affinity between Met at the 34^{th} residue and Trp at the 3^{rd} residue [M34, W3]V37M was −94.72±0.15 kJ mol⁻¹. By comparison, the affinity of [M37, W3]V37M was higher than that of [V37, W3]^{WT}, and the affinity of [M34, W3]^{V37M} was lower than [M34, W3]^{WT}. It can be inferred that mutated Met at the 37^{th} residue (M37) and Met at the 34^{th} residue contend for the affinity with Trp at the 3^{rd} residue, where the abnormally enhanced affinity [M37, W3]^{V37M} in V37M-mCx26 homomer brings about the conformational change of the wild-type channel portion, causing the NTH domain 37 (as shown in FIG. 4) to be abnormally attracted upwards towards the center of the channel from their original position tightly attached to the inner edge of the channel portion. Thus, the local variation with hydrophobicity change causes the tunnel shrunk, leading to the overall channel blocked. The channel shrinkage is shown in Table 3, which can be explained using average number of water molecules (i.e., the tunnel volume) in the V37M-mCx26 homomer model significantly less than that in the WT-mCx26 model.

### Build Knock-in Mice Animal Model

Using the recombinant engineering method developed by Copeland et al (PMID: 11352566, 17344389), and using the BAC clone (clone no. bMQ369p05 Geneservice™) with mouse Gjb2 gene region in the 129S7/AB2.2 BAC gene bank, a mutant gene targeting vector was established, and the BAC was transfected into Escherichia coli strain EL350 by electroporation. A subcloned 15.9 kb gene region was modified by a neomycin (neo) cassette inserted into PL452 plasmid to produce a V37M mutant. The aforementioned mutant gene targeting vector was cut apart with NotI restriction enzymes, and then transfected into R1 embryonic stem (ES) cells by electroporation. Next, the clone strains having resistance were screened out via G418 genemycin (240 μg mL⁻¹) and ganciclovir nucleoside analogue (2 μM), and identified using Southern blotting. By transfecting a cell harboring a target clone of plasmid that can temporarily express Cre recombinase, the retained neomycin (neo) cassette (with loxP sites at both ends) was excised within the cell. The established ES clone strain were confirmed using PCR screening and injected into blastocysts of a C57BL/6 line to generate chimera mice. These mice were raised under the C57BL/6 line gene background and subjected to subsequent experiments, and were cultivated to obtain Gjb2^{WT/WT} wild-type mice (hereinafter referred to as Gjb2^{WT/WT}), Gjb2^{WT/V37M} heterozygote knock-in mice (hereinafter referred to as Gjb2^{WT/V37M}) and Gjb2^{V37M/V37M} homozygote knock-in mice (hereinafter referred to as Gjb2^{V37M/V37M}). All animal experiments shall comply with the regulations for the care and use of experimental animals set by the National Taiwan University Hospital (approval no. 200700204).

### Hearing Test

The aforementioned animal model was anesthetized by intraperitoneal injection of sodium pentobarbital (35 mg kg⁻¹), and the hearing test was conducted in a soundproof and electrically insulating test room by using an evoked potential detection system (Smart EP 3.90, Intelligent Hearing Systems, Miami, FL) to measure the threshold of auditory brainstem response (ABR) of each mouse, and ABR of the mice was induced by click sounds with different intensities (loudness from 10 dB SPL to 130 dB SPL) of 8 kHz, 16 kHz, and 32 kHz.

Additionally, 13 Gjb2^{WT/WT} mice, 19 Gjb2^{WT/V37M} mice, and 30 Gjb2^{V37M/V37M} mice were subjected to the aforementioned hearing test, and the hearing thresholds measured at week 4, week 12, week 28, and week 44 were recorded.

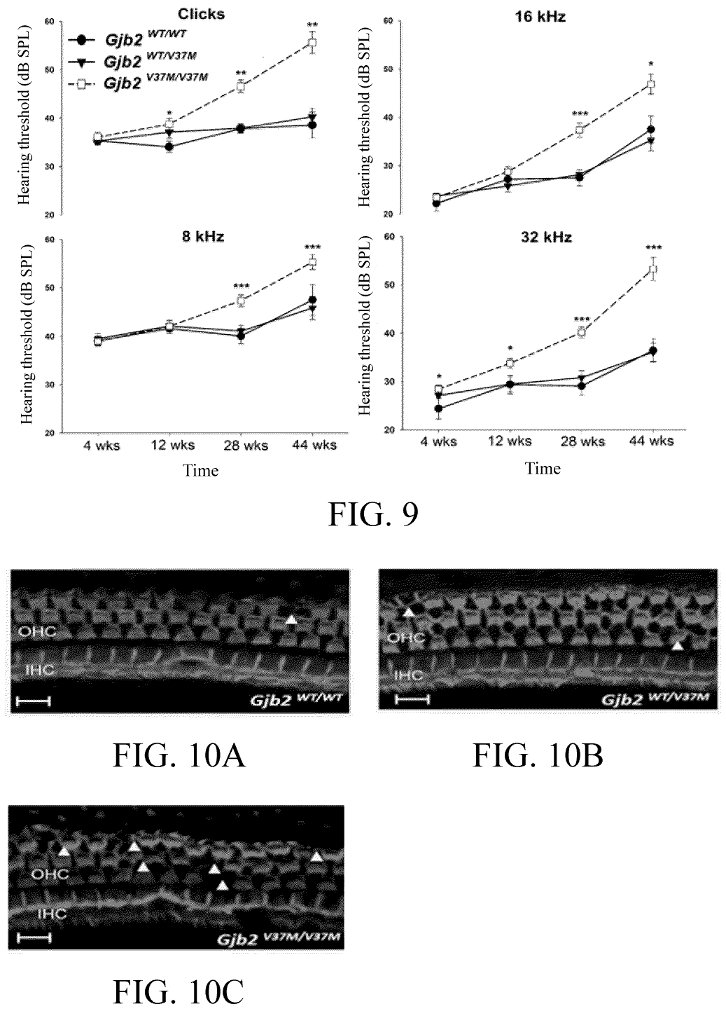

Referring to FIG. 9, FIG. 9 illustrates a comparison diagram of hearing test results for animal models in some embodiments, showing a distribution diagram of hearing thresholds in mice under the stimulation of click sounds at sound frequencies of 8 kHz, 16 kHz, 32 kHz. In FIG. 9, there was no significant difference in hearing threshold curves shown between Gjb2^{WT}WT mice and Gjb2^{WT}rv^{37M} mice under the stimulation of click sounds with different sound frequencies. However, Gjb2^{V37M/V37M} mice showed that their hearing deteriorated rapidly with the increase of time, especially at week 28 and week 44, and the hearing threshold measured from Gjb2^{V37M/V37M} mice was significantly higher than those of other two groups of mice, indicating that V37M in the animal experiments caused the hearing impairment in an autosomal recessive inheritance manner, which is consistent with the predicted results obtained by the prediction method herein.

### Inner Ear Cell Tissue Staining Test

The inner ear tissue of the aforementioned animal model was subjected to hematoxylin and eosin (H&E) staining and inner ear morphology was studied. Image analysis was performed on H&E stained tissues using the Nikon Optphot-2 microscope. In the study of inner ear morphology, staining was conducted with rhodamine-phalloidin (Molecular Probes, Eugene, OR, USA) at a dilution ratio of 1:100, and fluorescence images were captured by a laser scanning confocal microscope (Zeiss LSM 510, Germany).

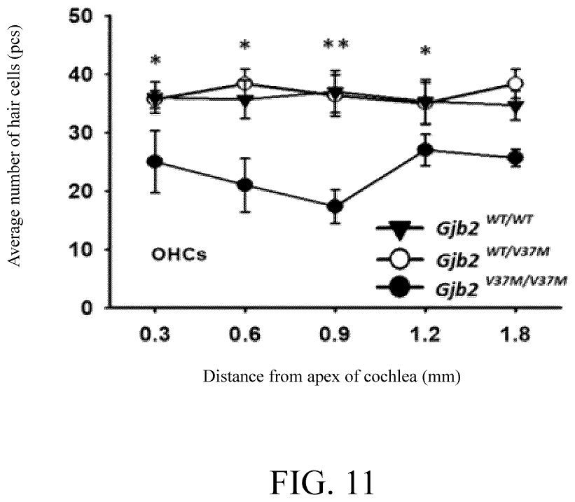

Referring to FIG. 10A to FIG. 11, FIG. 10A to FIG. 10C are tissue fluorescent staining images of middle turns of the organ of Corti from mouse cochleas of animal models Gjb2^{WT/WT}, Gjb2^{WT/V37M} and Gjb2^{V37M/V37M}, respectively. FIG. 11 is a comparison diagram of results of average number of hair cells in tissue fluorescence staining images of animal models Gjb2^{WT/WT}, Gjb2^{WT/V37M} and Gjb2^{V37M/V37M}.

In FIG. 10A, FIG. 10B and FIG. 10C, OHC represents outer hair cells, IHC represents inner hair cells, the scale bar in the drawing represents 10 μm, and the triangle symbols highlight the position of hair cell significant defects. FIG. 11 represents a graph of quantitative analysis results of fluorescent images of each group at week 44. The number of hair cells was computed per 100 m of fluorescent image according to the distance from the apex of the cochlea. According to the results shown in FIG. 10A to FIG. 10C, the tissue fluorescent staining image suggested the most severe hair cell defect in FIG. 10C compared to FIG. 10A and FIG. 10B. Furthermore, in FIG. 11 showing the comparison diagram of quantified hair cell distribution along the distance from the apex of the cochlea, Gjb2^{WT/WT} mice and Gjb2^{WT/V37M} mice have no significant difference, while the counterpart of Gjb2^{V37M/V37M} mice was significantly fewer than those of the Gjb2^{WT/WT} mice, indicating that the tissue staining results of are consistent with the hearing test results. The hearing deterioration of Gjb2^{V37M/V37M} mice is significantly faster than that of Gjb2^{WT/WT} mice and Gjb2^{WT/V37M} mice, and V37M in mice is identified to cause hearing impairment in an autosomal recessive inheritance manner. The above-mentioned results show that the inheritance pattern obtained from the animal model is consistent with the predicted result produced by the prediction system 10 herein.

Therefore, through the prediction system 10 and the method thereof, a user can predict the pathogenicity of the hereditary genetic variant based on physical quantities obtained from the computer simulation without obtaining preliminary clinical data, and efficiently predict the inheritance pattern of the pathogenicity, and the prediction system is suitable for predicting a pathogenic genetic variation related to the disorder of hereditary hearing loss.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A clinical pathologic data-free and computer-aided prediction method for genetic variation pathogenicity and an inheritance pattern thereof, wherein the prediction method is suitable for predicting a pathogenic genetic variation related to the disorder of hereditary hearing loss, the prediction method is executed by a computer arithmetic device, and the prediction method comprises:
a molecular dynamics simulation step: performing molecular dynamics simulations on channel protein configurations, wherein the channel protein configurations comprise a wild-type channel and mutated channels, and the mutated channels comprise a mutated homomeric channel and a plurality of mutated heteromeric channels;
a computing step: within a predetermined period in the respective computational simulation model, computing the number of ions passing through the wild-type channel to obtain a first ion number, and computing the number of ions passing through each mutated-channel to obtain a second ion number;
a determination step: determining a class of each mutated channel according to the first ion number and the second ion number, wherein when the second ion number is less than one-third of the first ion number, the mutated channel is determined as a blocking class; and when the second ion number is greater than or equal to half of the first ion number, the mutated channel is determined as a non-blocking class; and
a predicted result production step: producing a predicted result according to the classes of the mutated channels.

2. The prediction method according to claim 1, wherein after the molecular dynamics simulation step, the prediction method further comprises:
a sorting step: sorting the mutated heteromeric channels based on conformational potential energy obtained from the molecular dynamics simulation in which each mutated heteromeric channel reaches conformational equilibrium, wherein one of the mutated heteromeric channels with the lowest conformational potential energy is defined as a major mutated heteromeric channel; and
wherein in the predicted result production step, when both of the mutated homomeric channel and the major mutated heteromeric channel are determined as the blocking class, the predicted result indicates autosomal dominant inheritance.

3. The prediction method according to claim 1, wherein the molecular dynamics simulation step comprises:
a coordinate setting step: defining initial average positions of choline on an upper layer and a lower layer of a phospholipid bilayer as an upper leaflet line and a lower leaflet line, respectively, wherein each channel protein configuration is located in on the phospholipid bilayer;
a recording step: recording position changes of a plurality of ions in each channel protein configuration to obtain a plurality of moving trajectories; and
wherein in the computing step, the first ion number and the second ion number respectively represent numbers of ions whose trajectories intersect both the upper leaflet line and the lower leaflet line of the corresponding wild-type channel and mutant channel, wherein the ions completely traverse the respective channel protein configuration.

4. The prediction method according to claim 1, wherein after the molecular dynamics simulation step, the prediction method further comprises:
a candidate ion selection step:
storing a plurality of position marks for each of a plurality of ions in a time order by selecting each of a plurality of one-dimensional arrays on a first axis of a first two-dimensional tensor, wherein each of the plurality of position marks for each of the plurality of ions is selected from one element of group consisting of −a, 0, and b, wherein a and b are fixed positive integers, −a represents an index of first position, b represents an index of second position, and 0 represents an index of third position;
based on a plurality of index values along a second axis of the first two-dimensional tensor, executing:
for a current index value that is not yet selected, among a plurality of one-dimensional arrays along the second axis of the first two-dimensional tensor, generating an array of net values by subtracting an array indexed with the current index value from another array indexed with a next index value, and setting the array of net values as a one-dimensional array indexed with the current index value along a second axis of a second two-dimensional tensor; and repeating the above-mentioned steps for arrays of net values until all the index values are selected; and
in response to the presence of a candidate array of elements with an absolute value of a+b in a plurality of one-dimensional arrays along a first axis of the second two-dimensional tensor, selecting an ion with an index value corresponding to the candidate array from the ions as a candidate ion.

5. The prediction method according to claim 4, wherein in the computing step, the first ion number and the second ion number respectively represent numbers of ions whose trajectories, obtained from the molecular dynamics simulations of the corresponding wild-type channel and the mutated channels, intersect both the upper leaflet line and the lower leaflet line of the corresponding wild-type channel and the corresponding mutated channel, and completely traverse the respective channel.

6. The prediction method according to claim 1, wherein the channel protein configurations are categorized as at least one of connexin families, and each mutated heteromeric channel is composed of a wild-type protein monomer and a mutated protein monomer in any ratio.

7. The prediction method according to claim 1, wherein a monomer composing each mutated channel harbors at least one variation.

8. A clinical pathologic data-free and computer-aided prediction system for genetic variation pathogenicity and an inheritance pattern thereof, wherein the prediction system is suitable for predicting a pathogenic genetic variation related to the disorder of hereditary hearing loss, and the prediction system comprises:
a simulation module, configured to execute:
a molecular dynamics simulation step: performing molecular dynamics simulations on channel protein configurations, wherein the channel protein configurations comprise a wild-type channel and mutated channels, and the mutated channels comprise a mutated homomeric channel and a plurality of mutated heteromeric channels;
a processing module, configured to execute:
a computing step: within a predetermined period in the respective computational simulation model, computing the number of ions passing through the wild-type channel to obtain a first ion number, and computing the number of ions passing through each mutated channel to obtain a second ion number;
a determination step: determining a class of the mutated channel according to the first ion number and the second ion number, wherein when the second ion number is less than one-third of the first ion number, the mutated channel is determined as a blocking class; and when the second ion number is greater than or equal to half of the first ion number, the mutated channel is determined as a non-blocking class; and
a predicted result production step: producing a predicted result according to the classes of the mutated channels; and
a memory module, configured to store the channel protein configurations and the classes of the mutated channels.

9. The prediction system according to claim 8, wherein the processing module is configured to execute:
a candidate ion selection step:
storing a plurality of position marks for each of a plurality of ions in a time order by selecting each of a plurality of one-dimensional arrays on a first axis of a first two-dimensional tensor, wherein each of the plurality of position marks for each of the plurality of ions is selected from one element of group consisting of −a, 0, and b, wherein a and b are fixed positive integers, −a represents an index of first position, b represents an index of second position, and 0 represents an index of third position;
based on a plurality of index values along a second axis of the first two-dimensional tensor, executing:
for a current index value that is not yet selected, among a plurality of one-dimensional arrays along the second axis of the first two-dimensional tensor, generating an array of net values by subtracting an array indexed with the current index value from another array indexed with a next index value, and setting the array of net values as a one-dimensional array indexed with the current index value along a second axis of a second two-dimensional tensor; and repeating the above-mentioned steps for arrays of net values until all the index values are selected; and
in response to the presence of a candidate array of elements with an absolute value of a+b in a plurality of one-dimensional arrays along a first axis of the second two-dimensional tensor, selecting an ion with an index value corresponding to the candidate array from the ions as a candidate ion.

10. An ion selection method, executed by a processing unit, wherein the ion selection method comprises:
storing a plurality of position marks for each of a plurality of ions in a time order by selecting each of a plurality of one-dimensional arrays on a first axis of a first two-dimensional tensor, wherein each of the plurality of position marks for each of the plurality of ions is selected from one element of the group consisting of −a, 0, and b, wherein a and b are fixed positive integers, −a represents an index of first position, b represents an index of second position, and 0 represents an index of third position;
based on a plurality of index values of a second axis along the first two-dimensional tensor, executing:
for a current index value that is not yet selected, among a plurality of one-dimensional arrays along the second axis of the first two-dimensional tensor, generating an array of net values by subtracting an array indexed with the current index value from an array indexed with a next index value, and setting the array of net values as a one-dimensional array indexed with the current index value along a second axis of a second two-dimensional tensor; and repeating the above-mentioned steps for arrays of net values until all the index values are selected; and
in response to the presence of a candidate array of elements with an absolute value of a+b in a plurality of one-dimensional arrays along a first axis of the second two-dimensional tensor, selecting an ion with an index value corresponding to the candidate array from the ions as a candidate ion.

11. The ion selection method according to claim 10, wherein the one-dimensional arrays along the first axis of the first two-dimensional tensor are a plurality of column vectors of the first two-dimensional tensor; the one-dimensional arrays along the first axis of the second two-dimensional tensor are a plurality of column vectors of the second two-dimensional tensor.

12. The ion selection method according to claim 10, wherein a is selected as 1, and b is selected as 1.

13. The ion selection method according to claim 10, wherein the first position is a position between a lower leaflet line and a midline of a phospholipid bilayer, the second position is a position between an upper leaflet line and the midline of the phospholipid bilayer, and the third position is another position other than the first position and the second position.

14. An ion selection system, comprising a processing unit, and configured to execute the following steps:
storing a plurality of position marks for each of a plurality of ions in a time order by selecting each of a plurality of one-dimensional arrays on a first axis of first two-dimensional tensor, wherein each of the plurality of position marks for each of the plurality of ions is selected one element of the group consisting of −a, 0, and b, wherein a and b are fixed positive integers, −a represents an index of first position, b represents an index of second position, and 0 represents an index of third position;
based on a plurality of index values along a second axis of the first two-dimensional tensor, executing:
for a current index value that is not yet selected, among a plurality of one-dimensional arrays along the second axis of the first two-dimensional tensor, generating an array of net values by subtracting an array indexed with the current index value from an array indexed with a next index value, and setting the array of net values as a one-dimensional array indexed with the current index value along second axis of a second two-dimensional tensor; and repeating the above-mentioned steps for arrays of net values until all the index values are selected; and
in response to the presence of a candidate array of elements with an absolute value of a+b in a plurality of one-dimensional arrays along a first axis of the second two-dimensional tensor, selecting an ion with an index value corresponding to the candidate array from the ions as a candidate ion.
